(19) United States
(12) Patent Application Publication
(10) Pub. No.: US 2005/0267359 A1
(43) Pub. Date: Dec. 1, 2005

(54) SYSTEM, METHOD, AND ARTICLE OF MANUFACTURE FOR GUIDING AN END EFFECTOR TO A TARGET POSITION WITHIN A PERSON

(75) Inventors: Mohammed Moin Hussaini, Baltimore, MD (US); Thomas Foo, Potomac, MD (US)

Correspondence Address: CANTOR COLBURN, LLP, 55 GRIFFIN ROAD SOUTH, BLOOMFIELD, CT, 06002, US

(73) Assignee: GENERAL ELECTRIC COMPANY, Schenectady, NY (US)

(21) Appl. No.: 10/709,783

(22) Filed: May 27, 2004

(51) Int. Cl. A61B 5/05

(52) U.S. Cl. 600/423; 600/434

(57) ABSTRACT

A system, method, and article of manufacture for guiding an end effector to a target position within a person are provided. The method includes generating a plurality of digital images of an interior anatomy of the person when the person has a predetermined respiratory state. The method further includes indicating a skin entry position on at least one of the digital images. The method further includes indicating the target position on at least one of the digital images. The method further includes determining a trajectory path based on the skin entry position and the target position. Finally, the method includes moving the end effector along the trajectory path toward the target position when the person has substantially the predetermined respiratory state.

### BACKGROUND OF INVENTION

The invention relates to a system and a method for guiding an end effector to a target position with a person.

Robotic systems have been developed to guide biopsy and ablation needles within a person. However, the placement of such needles within the abdomen of the person can be very difficult due to the respiratory motion of the person. In particular, during respiratory motion of the person, a target position within the abdomen of the person will move. Thus, even if the needle is initially moved along a predetermined end effector trajectory, the needle may not reach the target position due to the movement of the target position within the abdomen of the person.

Thus, the inventors herein have recognized that a need exists for an improved system that overcomes the aforementioned drawbacks when guiding an end effector to a target position within the person.

### SUMMARY OF INVENTION

A method for guiding an end effector to a target position within a person in accordance with an exemplary embodiment is provided. The method includes generating a plurality of digital images of an interior anatomy of the person when the person has a predetermined respiratory state. The method further includes indicating a skin entry position on at least one of the digital images. The method further includes indicating the target position on at least one of the digital images. The method further includes determining a trajectory path based on the skin entry position and the target position. Finally, the method includes moving the end effector along the trajectory path toward the target position when the person has substantially the predetermined respiratory state.

A system for guiding an end effector to a target position within a person in accordance with another exemplary embodiment is provided. The system includes a respiratory monitoring device for monitoring a respiratory state of the person. The system further includes a scanning device configured to scan an interior anatomy of the person when the person has a predetermined respiratory state to generate scanning data. The system further includes a first computer generating a plurality of digital images based on the scanning data. The system further includes a second computer configured to display the plurality of digital images, the second computer is further configured to allow an operator to indicate a skin entry position on at least one of the digital images. The second computer is further configured to allow the operator to indicate the target position on at least one of the digital images. The second computer is further configured to determine a trajectory path based on the skin entry position and the target position. Finally, the system includes an end effector insertion device having the end effector adapted to be inserted into the person, the second computer inducing the end effector insertion device to move the end effector along the trajectory path toward the target position when the person has substantially the predetermined respiratory state.

A system for guiding an end effector to a target position within a person in accordance with another exemplary embodiment is provided. The system includes a respiratory monitoring device for monitoring a respiratory state of the person. The system further includes a scanning device configured to scan an interior anatomy of the person when the person has a predetermined respiratory state to generate scanning data. The system further includes a first computer generating a plurality of digital images based on the scanning data. The first computer is further configured to display the plurality of digital images. The first computer is further configured to allow an operator to indicate a skin entry position on at least one of the digital images. The first computer is further configured to allow the operator to indicate the target position on at least one of the digital images. The first computer is further configured to determine a trajectory path based on the skin entry position and the target position. Finally, the system includes an end effector insertion device having the end effector adapted to be inserted into the person. The first computer induces the end effector insertion device to move the end effector along the trajectory path toward the target position when the person has substantially the predetermined respiratory state.

An article of manufacture in accordance with another exemplary embodiment is provided. The article of manufacture includes a computer storage medium having a computer program encoded therein for guiding an end effector to a target position within a person. The computer storage medium includes code for generating a plurality of digital images of an interior anatomy of the person when the person has a predetermined respiratory state. The computer storage medium further includes code for indicating a skin entry position on at least one of the digital images. The computer storage medium further includes code for indicating the target position on at least one of the digital images. The computer storage medium further includes code for determining a trajectory path based on the skin entry position and the target position. Finally, the computer storage medium includes code for moving the end effector along the trajectory path toward the target position when the person has substantially a predetermined respiratory state.

A method for guiding an end effector to a target position within a person in accordance with another exemplary embodiment is provided. The method includes monitoring a respiratory state of a person during at least one respiratory cycle. Finally, the method includes moving an end effector along a trajectory path toward the target position in the person when the person has substantially a predetermined respiratory state.

### BRIEF DESCRIPTION OF DRAWINGS

FIG. 1 is a schematic of an operatory room containing an end effector positioning system in accordance with an exemplary embodiment.

FIG. 2 is a schematic of the end effector positioning system of FIG. 1.

FIG. 3 is in an enlarged schematic of a portion of the end effector positioning system of FIG. 2.

FIG. 4 is a schematic of a robotic end effector positioning device and a passive arm utilized in the end effector positioning system of FIG. 2.

FIGS. 5-7 are schematics of an end effector driver used in the robotic end effector positioning device of FIG. 4.

FIG. 8 is a signal schematic indicative of respiratory motion of a person.

FIG. 9 is a signal schematic indicative of a predetermined respiratory state of the person.

FIG. 10 is a diagram of three coordinate systems utilized by the end effector positioning system of FIG. 1.

FIGS. 11-15 are schematics of computer windows utilized by the end effector positioning system of FIG. 1.

FIGS. 16-18 are flowcharts of a method for guiding an end effector to a target position within a person.

### DETAILED DESCRIPTION

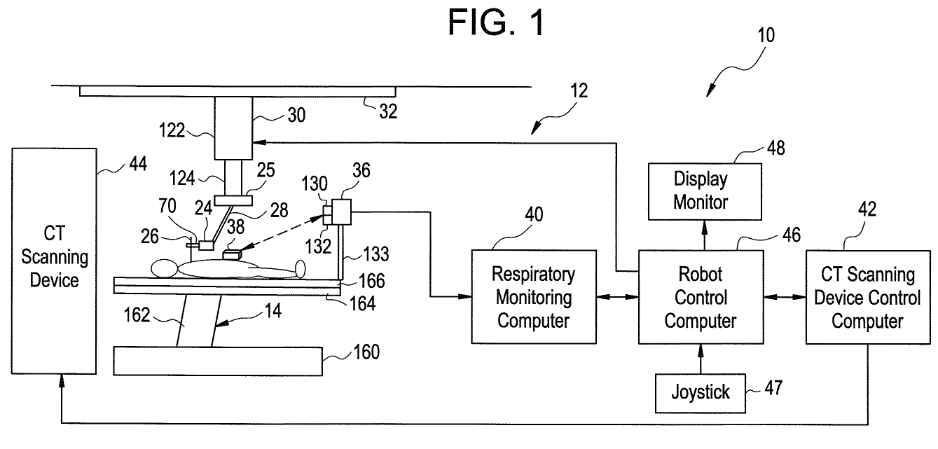

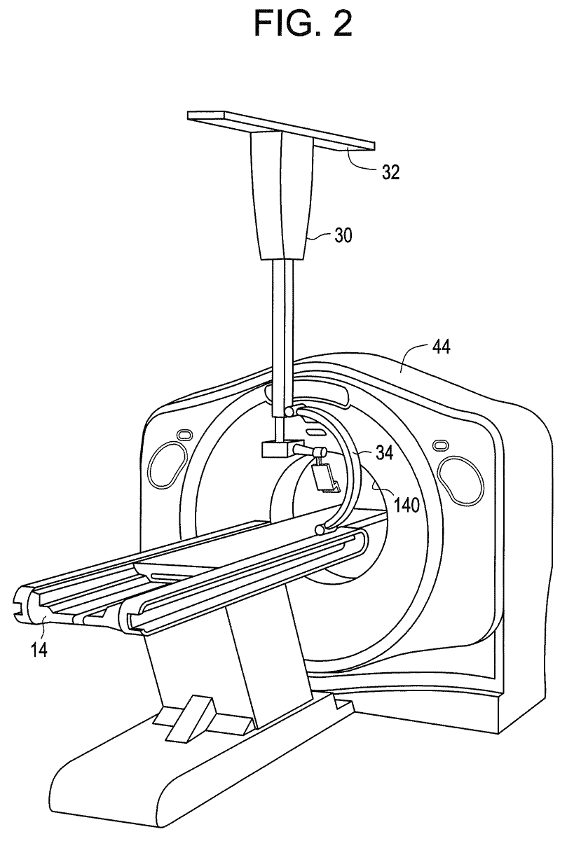

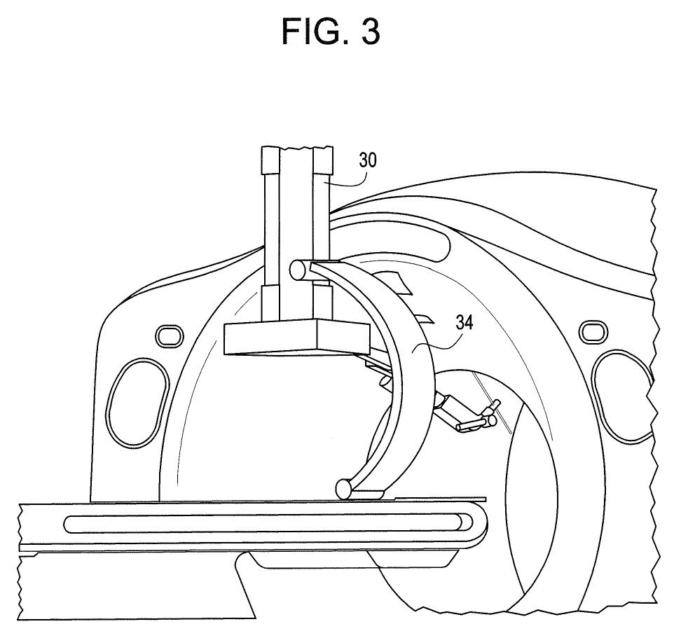

Referring to FIGS. 1 and 2, an operatory room 10 having an end effector positioning system 12 and an operatory table 14 is illustrated. The end effector positioning system 12 is provided to guide an end effector within a person lying on the table 14, to a predetermined position, as will be explained in greater detail below. The end effector in the illustrated embodiment comprises an ablation needle. It should be understood, however, that the end effector can be any tool or device that can be inserted within an interior of a person including a hypodermic needle, a biopsy needle, a steerable needle, and an orthoscopic tool, for example.

The end effector positioning system 12 includes a robotic end effector positioning device 24, an end effector driver 70, a linear positioning device 25, a passive arm 28, an overhead support 30, a rail support 32, a coupling bracket 34, an infrared respiratory measurement device 36, a position reflector 38, a respiratory monitoring computer 40, a CT scanning device control computer 42, a computerized tomography (CT) scanning device 44, a robot control computer 46, a joystick 47, and a display monitor 48.

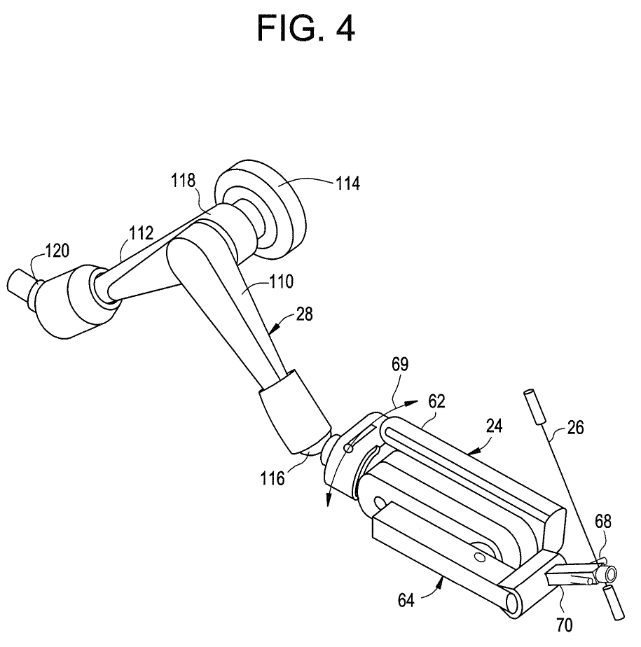

Referring to FIG. 4, the linear positioning device 25 is operably coupled to the overhead support 30 and the passive arm 28. The linear positioning device 25 is provided to linearly move the robotic end effector about 3 axes to position device 24 to a desired linear position. In the illustrated embodiment, the linear positioning device 25 comprises an XYZ Stage manufactured by Danaher Precision systems of Salem, N.H.

The robotic end effector positioning device 24 is provided for orienting the end effector driver 70 so that an end effector 26 can be positioned coincident with a desired trajectory. The robotic end effector positioning device 24 is electrically coupled to the robot control computer 46 and moves responsive to signals received from the computer 46. As shown, the robotic end effector positioning device 24 includes a housing portion 62 and a housing portion 64. As shown, the robotic end effector positioning device 24 is operably coupled to the end effector driver 70.

The housing portion 64 is provided to house a motor (not shown) therein that has a shaft operably coupled to a joint 116 of the passive arm 28. The motor is configured to rotate the robotic end effector positioning device 24 as shown by the arrow 69 for positioning the end effector 26 to a desired position. The housing portion 64 is operably coupled to the housing portion 62 and is provided to house a motor for driving components in the end effector driver 70 to linearly move the end effector 26.

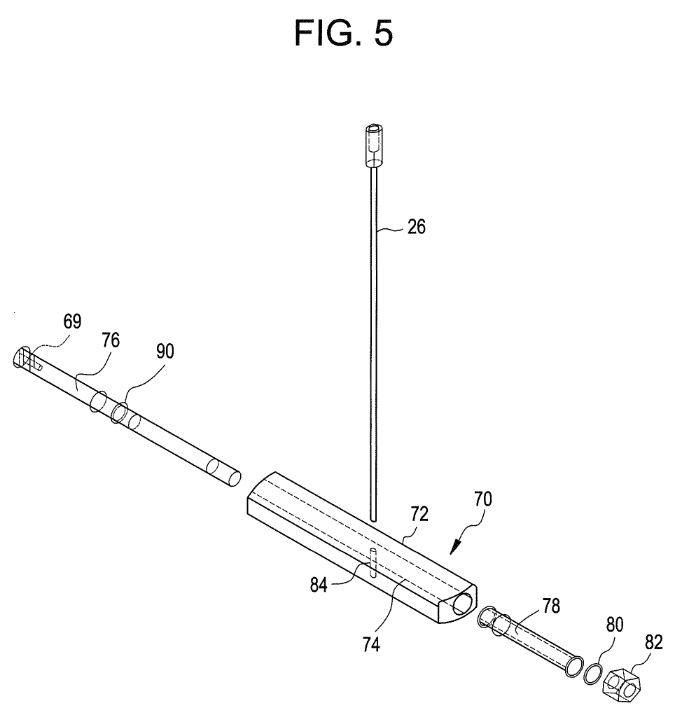

Referring to FIGS. 4-7, the end effector driver 70 is provided to linearly move the end effector 26 into a person. The end effector driver 70 includes housing portion 72 operably coupled to the end effector 26. An input shaft 76 is driven by a DC motor (not shown), which is located in the housing portion 64. The housing portion 72 can be constructed of acrylic or another radiolucent material. The housing portion 72 defines a first rimmed bore 74 extending thereacross and configured to slidingly receive input shaft 76 and axial loading bushing 78 therein. The bushing 78 slides over the input shaft 76, and is loaded through an O-ring 80 with a nut 82. The housing portion 72 further defines a second rimmed bore 84 therein extending transversely tangential to the first rimmed bore 74 within the housing portion 72. The input shaft 76, the bushing 78, and the nut 82 can be constructed of acrylic or another radiolucent material. The input shaft 76 is further coupled by a driven end 69 to the DC motor, and at another end thereof to the nut 82. By coupling the input shaft 76 to the nut 82 at the same rotational speed as the input shaft 76, the bushing 78 is driven by loading O-ring 80 with the nut 82.

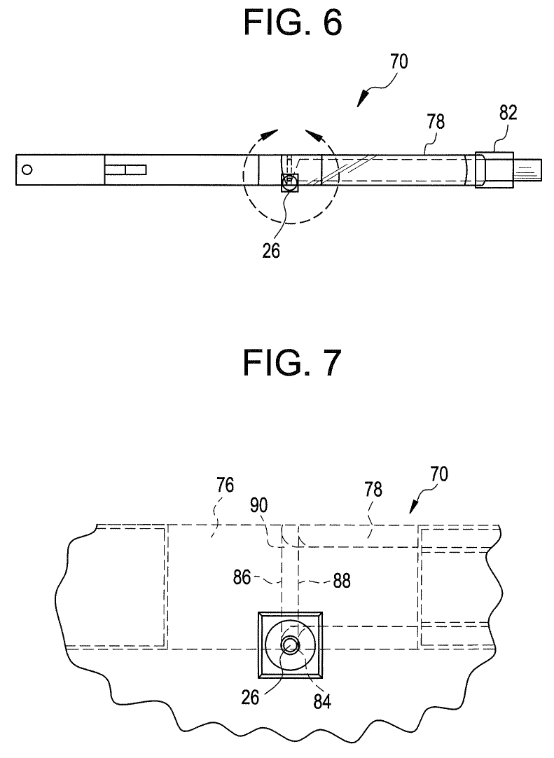

Referring to FIGS. 6 and 7, the end effector 26 slides in the second rimmed bore 84 of the housing portion 72, and as a result, is pressed between a contact face 86 of the input shaft 76 and a contact face 88 of the bushing 78. The contact face 88 corresponds to one of the two ends of the bushing. The contact faces 86 and 88 impart an axial force to the end effector 26 corresponding to the transmission friction force between the contact faces and the end effector 26. Further, a fillet 90 may be placed at the base of the contact face 86 of the input shaft 76.

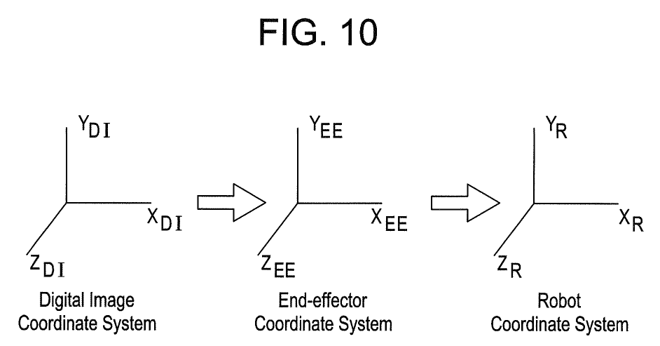

Referring to FIGS. 4 and 10, a fiducial component 68 extended from the end effector driver 70 is provided to correlate the robot coordinate system to the digital image coordinate system, as will be explained in greater detail below. The fiducial component 68 is generally v-shaped with first and second legs of the component 68 extending from opposite sides of the housing of the needle driver 70.

The passive arm 28 is provided to hold the robotic end effector positioning device 24. As shown, the passive arm 28 includes an arm portion 110, an arm portion 112, a clamping portion 114, and ball joints 116, 118, 120. The robotic end effector positioning device 24 is attached to the arm portion 110 via the ball joint 116 disposed therebetween. The arm portion 110 is operably coupled to the arm portion 112 via the ball joint 118. When the clamping portion 114 is loosened, the arm portion 112 and the arm portion 110 can move relative to each other via the ball joint 118, and the ball joints 116 and 120 are also loosened. When the clamping portion 114 is tightened, the arm portion 110 is fixed relative to the arm portion 112 and the ball joints 116 and 120 are locked into a predetermined position. The passive arm 28 is operably coupled to the overhead support 30 via the joint 120.

Referring to FIG. 1, the overhead support 30 is provided to hold the passive arm 28 and the robotic end effector positioning device 24 suspended above a person. The overhead support 30 includes a support portion 122 and a support portion 124. Support portion 124 is telescopically received within the support portion 122. Thus, the support portion 124 can be raised or lowered relative to the support portion 122 to initially position the end effector 26 to a desired skin entry point on the person. As shown, the overhead support 30 is operably attached to a rail support 32 that is further attached to a ceiling of the operatory room 10.

The rail support 32 is provided to allow movement of the robotic end effector positioning device 24 linearly with respect to a person. Referring to FIG. 2, the overhead support 30 can be coupled via a coupling bracket 34 to a movable section of the table 14. Accordingly, when the table 14 and the person lying thereon move linearly with respect to the CT scanning device 44, the overhead support 30 moves linearly via the rail support 32 to allow the robotic end effector positioning device 24 to remain at a fixed position relative to the person during such movement.

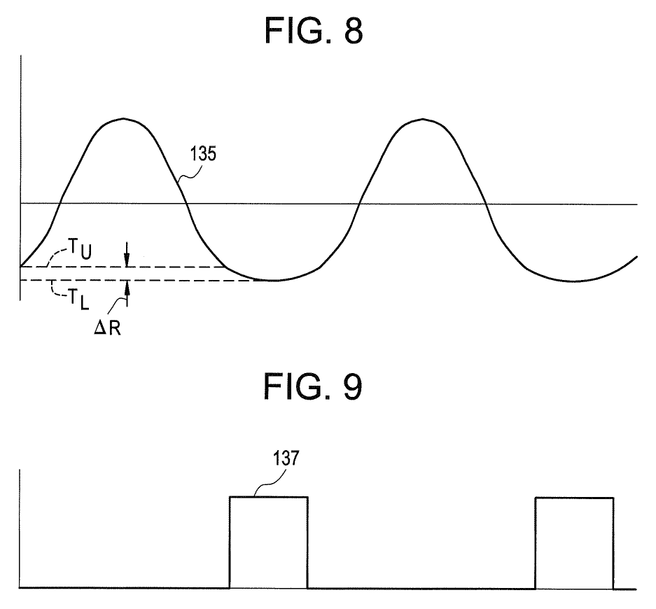

Referring to FIGS. 1 and 8, the infrared respiratory measurement device 36 is provided to measure a respiration state of the person lying on the table 14. The infrared respiratory measurement device 36 includes infrared transmitter 130 and infrared detector 132. As shown, the infrared respiratory measurement device 36 can be mounted on a stand 133 operably coupled to the table 14. The infrared transmitter 130 directs an infrared beam towards a reflector 38 positioned on a chest of the person. The infrared beam is thereafter reflected from the infrared reflector 38 towards the infrared detector 132. The infrared detector 132 receives the reflected infrared beam and generates a signal 135 that is indicative of the position of the person's chest responsive to the reflected infrared beam. The position of the chest of the person is further indicative of the respiratory state of the person.

The respiratory monitoring computer 40 is provided to receive the signal 135 indicative of the respiratory state of the person. The computer 40 is further configured to determine when the amplitude of the signal 135 is within a predetermined range ΔR having an upper threshold (T_{U}) and a lowest threshold (T_{L}). When the signal 135 is within the predetermined range ΔR indicative of a predetermined respiratory state, the computer 40 generates a gating signal 137 that is transmitted to the robot control computer 46. As will be described in greater detail below, the robot control computer 46 will linearly move the end effector 26 into the person when the gating signal 137 is at a high logic level. Further, when the gating signal 137 is not at a high logic level, the robot control computer will stop linear movement of the end effector 26.

Referring to FIGS. 1 and 2, the computerized tomography (CT) scanning device 44 is provided to take a plurality of CT digital images of an interior anatomy of the person within a predetermined scanning range. As shown, CT scanning device 44 includes an opening 140 in which a portion of the table 14 and a person can extend therethrough. The predetermined scanning range of the CT scanner 44 is within the opening 140. The plurality of CT digital images is utilized by an operator of the end effector positioning system 12 to determine (i) a skin entry point for the end effector 26, and (ii) a target location within the person where a tip of the end effector 26 is to be positioned. The CT scanning device 44 is operably coupled to the CT scanning device control computer 42. It should be noted that the end effector positioning system 12 could be utilized with other types of medical imaging devices instead of the CT scanning device 44, such as a magnetic resonance imaging (MRI) device, an ultrasound imaging device, or an x-ray device, for example.

The CT scanning device control computer 42 is provided to control the operation of the CT scanning device 44. In particular, the computer 42 induces the device 44 to scan a person to generate scanning data. Thereafter, the computer 42 processes the scanning data and generates a plurality of digital images of an internal anatomy of a person from the scanning data. Thereafter, the robot control computer 46 can query the computer 42 to induce the computer 42 to transmit the digital images to the robot control computer 46.

The robot control computer 46 is provided to control the movement of the end effector 26 by controlling movement of the robotic end effector positioning device 24 and the linear positioning device 25. The robot control computer 46 is electrically coupled to the respiratory monitoring computer 40 receiving the gating signal 137. The robot control computer 46 is further electrically coupled to the computer 42 for receiving the plurality of CT digital images of the person. Further, the computer 46 is electrically coupled to the robotic end effector positioning device 24. An operator of the computer 46 can display the plurality of CT digital images in computer windows on a display monitor 48. The operator can also select a skin entry point on a person and a target position within the person via touchscreen computer windows.

The table 14 is provided to support a person and to further move the person within the scanning region of the CT scanning device 44. The table 14 includes a base 160, a vertical support member 162, a fixed table top portion 164, and a movable table top portion 166. As shown, the fixed table top portion 164 is supported by the vertical support member 162. The support member 162 is further fixedly attached to the base 160. The movable table top portion 166 can be moved linearly with respect to the fixed table top portion 164. As discussed above, a coupling bracket 34 is disposed between the passive arm 28 and the movable table top portion 166 to maintain a relative position between the robotic end effector positioning device 24 and the person, when the person is being moved into the scanning region of the CT scanning device 44.

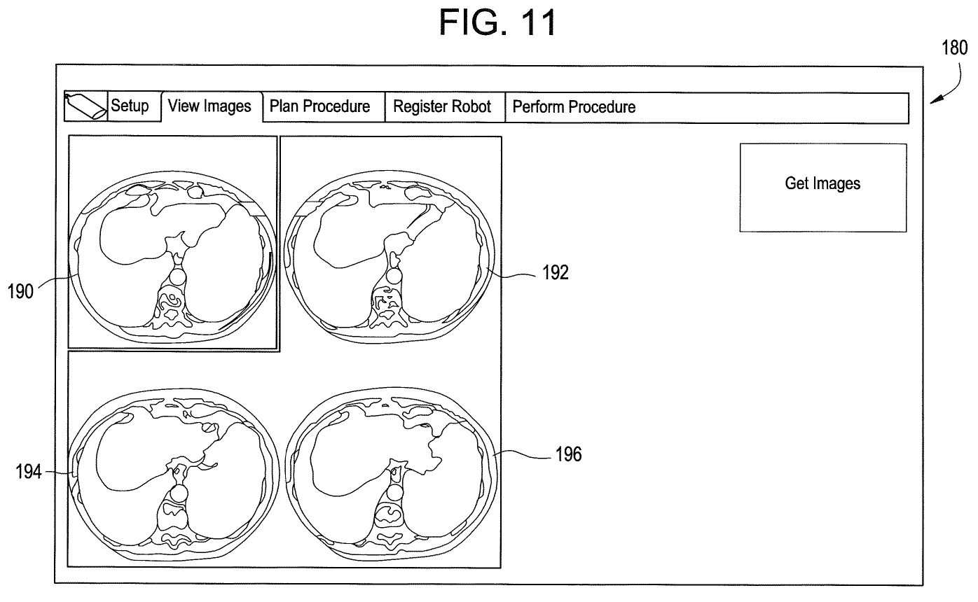

Before providing a detailed explanation of the method for guiding movement of the end effector 26 within a person from a skin entry point to a target point, a brief overview of the control windows utilized by the robot control computer 46 for determining an end effector trajectory and for controlling the robotic end effector positioning device 24 will be explained. Referring to FIG. 11, a computer window 180 that is generated by the robot control computer 46 on the display monitor 48 is illustrated. The computer window 180 includes several command icons including (i) a “Setup” icon, (ii) a “View Images” icon, (iii) a “Plan Procedure” icon, (iv) a “Register Robot” icon, and (v) a “Perform Procedure” icon, which will be explained in greater detail below.

When an operator of the robot control computer 46 selects the “Setup” icon, the operator is allowed to input an end effector movement speed that will be used when guiding the end effector 26 into the person.

When the operator of the robot control computer 46 selects the “View Images” icon, the computer 46 displays the computer window 180. When an operator selects the “Get Images” icon, the computer 46 queries the CT scanning device control computer 42 to obtain a plurality of digital images obtained from the CT scanning device 44. Thereafter, the robot control computer displays a predetermined number of the digital images in the computer window 180. For example, the digital images 190, 192, 194, 196 can be displayed in the computer window 180. The digital images 190, 192, 194, 196 represents cross-sectional images of an abdomen of a person.

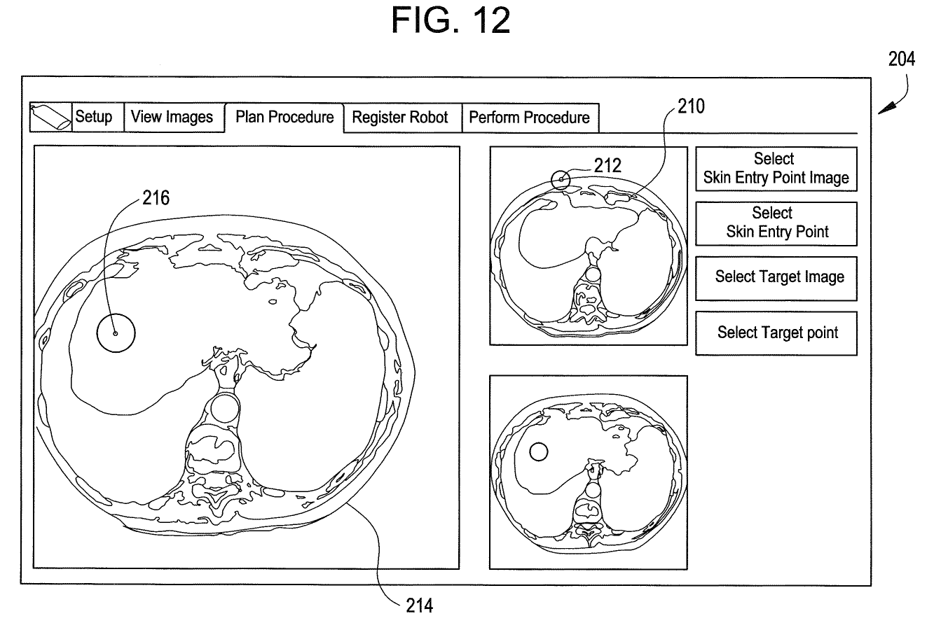

Referring to FIG. 12, when the operator of the robot control computer 46 selects of the “Plan Procedure” icon, the computer 46 displays the computer window 204. The computer window 204 is provided to allow the operator to select a skin entry point where the end effector 26 will be initially inserted into the person. Further, the window 204 is provided to allow the operator to select a target point within the person where the tip of end effector 26 is to be moved. As shown, the window 204 includes the following selection icons: (i) the “Select Skin Entry Point Image” icon, (ii) the “Select Skin Entry Point” icon, (iii) the “Select Target Image” icon, and (iv) the “Select Target Point” icon.

The “Select Skin Entry Point Image” icon allows the operator to view a plurality of digital images to determine a specific digital image that has a desired skin entry area for the end effector 26. As shown, the operator can select an digital image 210 that has a desired skin entry area.

The “Select Skin Entry Point” icon allows an operator to select a point on a specific digital image for specifying the skin entry point for the end effector 26. As shown, the operator can select a skin entry point 212 on the digital image 210.

The “Select Target Image” icon allows an operator to view a plurality of digital images to select a specific target digital image that has a desired target area for a tip of the end effector 26. As shown, the operator can select a digital image 214 that has a desired target area.

The “Select Target Point” icon allows an operator to select a point on a specific target digital image for specifying the target point for the end effector 26. As shown, the operator can select a target point 216 on the digital image 214.

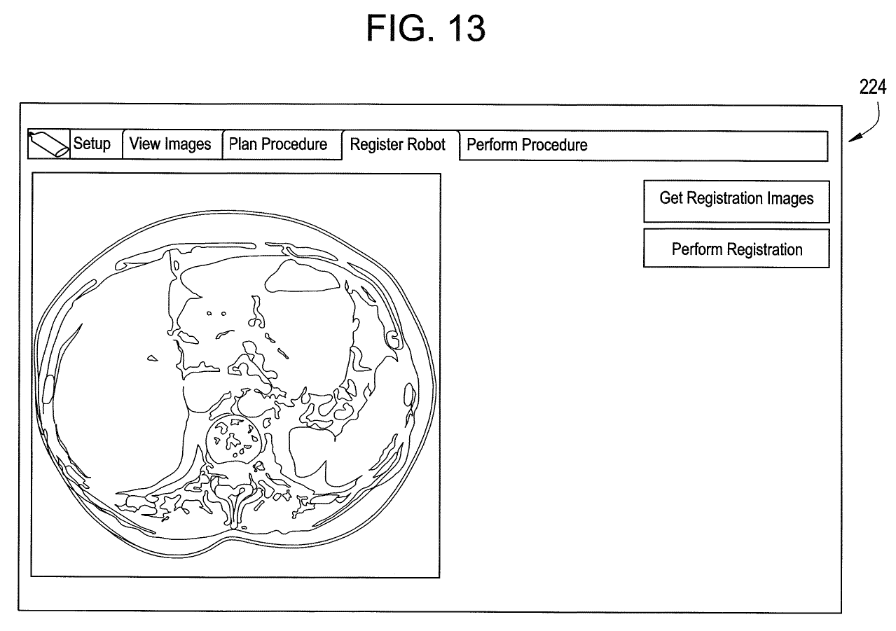

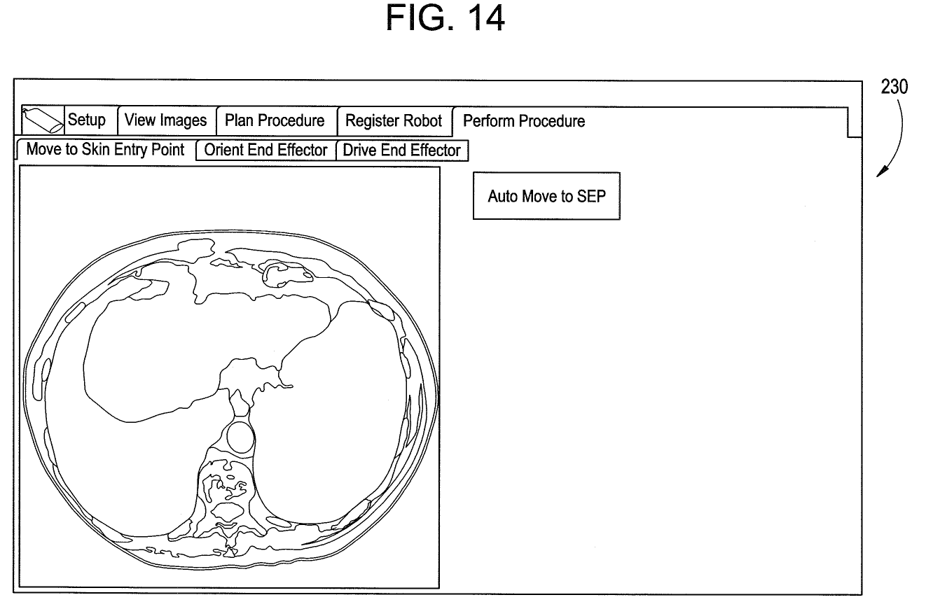

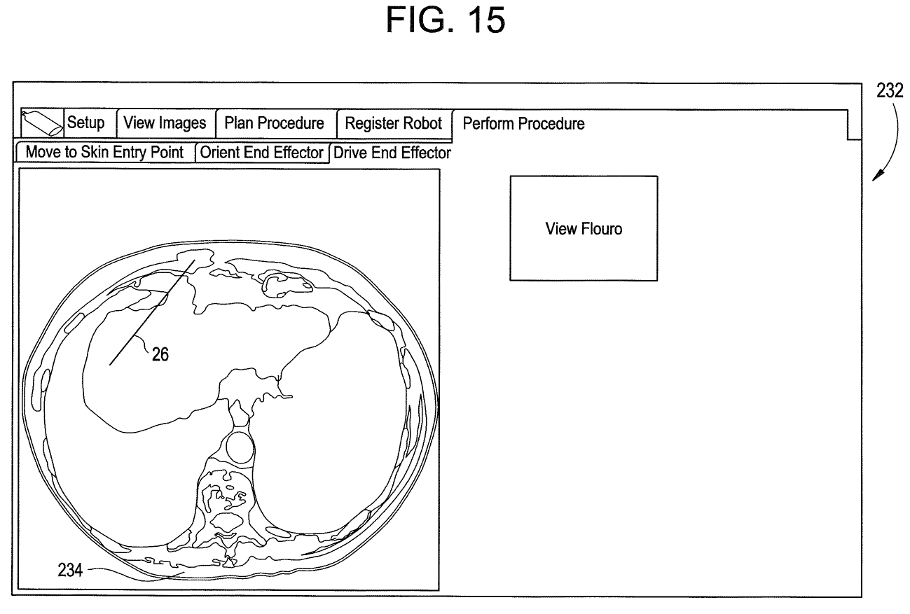

Referring to FIGS. 10 and 13, when an operator selects the “Register Robot” icon, the robot control computer 46 generates the computer window 224 on the display monitor 48 and retrieves digital images from the CT scanning device control computer 42. The “Perform Registration” icon enables the operator to command the robotic end effector positioning device 24 to a desired position to locate the end effector 26 at points identified in the digital or CT image coordinate system (e.g., skin entry point and target point). In particular, the operator is allowed to manually move the overhead support 30 and the robotic end effector positioning device 24 to grossly position the tip of the end effector 26 in the vicinity of the desired skin entry point. Prior to a pre-operative scan of a person, the digital image coordinate system is related to the fixed robot coordinate system so that the robotic end effector positioning device 24 can be commanded to move the end effector 26 to points specified in the digital image coordinate system. This process has six steps: (i) generate a digital image of the fiducial component 68 that is affixed in a known position and orientation with respect to the end effector 26, (ii) determine the position and orientation of the end effector 26 relative to the digital image coordinate system using the digital image, (iii) from the position and orientation determined at the prior step, construct a first homogeneous coordinate transformation matrix (e.g., homogenous transform) that defines the spatial relationship between the end effector coordinate system and the digital image coordinate system, (iv) determine the position and orientation of the end effector 26 relative to the robot reference frame via the robot kinematics properties, (v) from the position and orientation determined at the prior step, construct a second homogeneous coordinate transformation matrix that defines the spatial relationship between the end effector coordinate system and the robot coordinate system, (vi) multiply the first and second homogenous coordinate transformation matrices to obtain a third coordinate transformation matrix that allows the operator to specify robot movement in the digital image coordinate system.

Referring to FIG. 14, when an operator of the robotic control computer 46 selects the “Perform Procedure” icon, the computer 46 displays the computer window 230 on the display monitor 48. The window 230 includes the following command icons: (i) the “Move to Skin Entry Point” icon, (ii) the “Orient End Effector” icon, and (iii) the “Drive End Effector” icon.

When an operator selects the “Move to Skin Entry Point” icon the “Auto Move to Skin Entry Point” icon is displayed. Thereafter, when the operator selects the “Auto Move to Skin Entry Point” icon, the linear positioning device 25 moves the tip of the end effector from the registration position to the desired skin entry point upon actuation of the joystick 47.

When an operator selects the “Orient End effector” icon, and the operator actuates the joystick 47, the robotic end effector positioning device 24 orientates the tip of the end effector 26 along a calculated trajectory path based upon the selected skin entry point and the target point.

When an operator selects the “Drive End effector” icon and actuates the joystick 47, the robotic end effector positioning device 24 commences linearly moving the tip of the end effector 26 from the skin entry point to the target point when a predetermined respiratory state is obtained. Further, the robot control computer 46 will display a computer window 232 which includes a “View Fluoro” icon. When the operator selects the “View Fluoro” icon, a realtime digital image 234 can be displayed to allow the operator to view the travel path of the end effector 26 within the person.

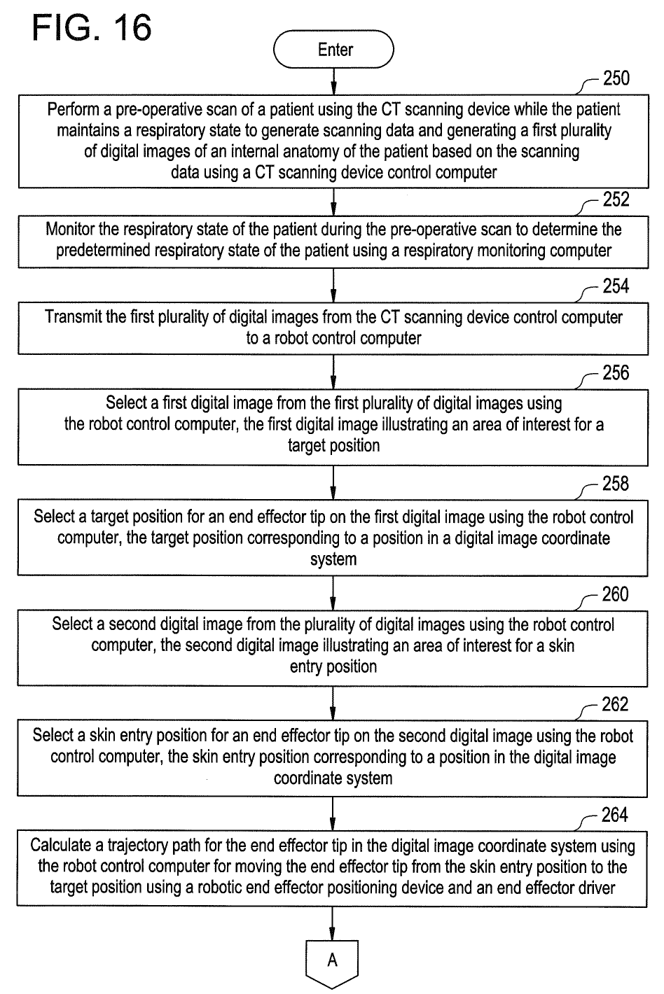

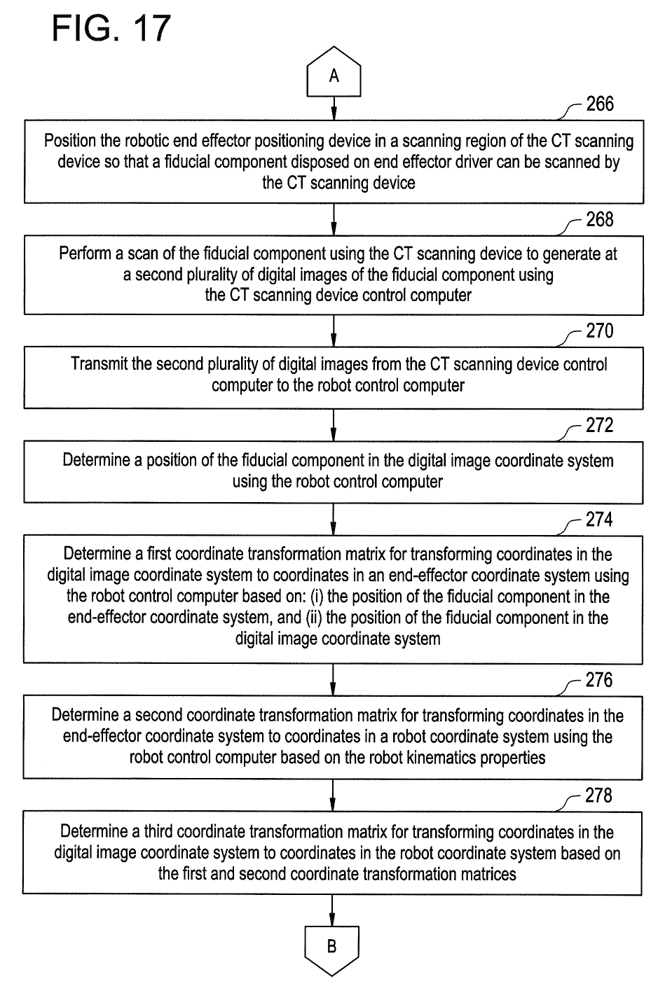

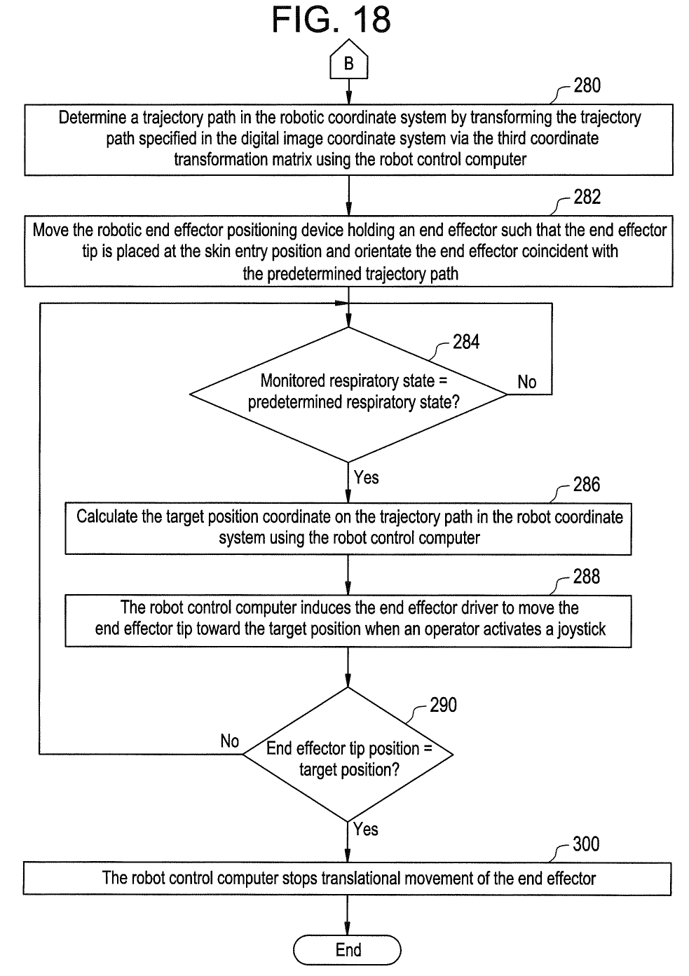

Referring to FIG. 16, a method for guiding an end effector 26 from a skin entry point to a target position with the person will now be explained.

At step 250, the CT scanning device 44 performs a pre-operative scan of a person, while the person maintains a respiratory state and generates scanning data. The CT scanning device control computer generates a first plurality of digital images of an internal anatomy of the person based on the scanning data. It should be noted that during the pre-operative scan, the person substantially maintains a predetermined respiratory state, such as a full-inhalation position or a full exhalation position for example.

At step 252, a respiratory monitoring computer 40 monitors the respiratory state of the person during the pre-operative scan to determine the predetermined respiratory state of the person. In particular, the respiratory monitoring computer 40 receives the gating signal 137 indicative of the respiratory state of the person.

At step 254, the CT scanning device control computer 42 transmits the first plurality of digital images to the robot control computer 46.

At step 256, an operator of the robot control computer 46 selects a first digital image from the first plurality of digital images. The first digital image illustrates an area of interest for a target position.

At step 258, an operator of the robot control computer 46 selects a target position for an end effector tip on the first digital image. The target position corresponds to a position in a digital image coordinate system.

At step 260, an operator of the robot control computer 46 selects a second digital image from the plurality of digital images. The second digital image illustrates an area of interest for a skin entry position.

At step 262, an operator of the robot control computer 46 selects a skin entry position for an end effector tip on the second digital image. The skin entry position corresponds to a position in the digital image coordinate system.

At step 264, the robot control computer 46 calculates a trajectory path for an end effector tip in the digital image coordinate system for moving the end effector tip from the skin entry position to the target position using a robotic end effector positioning device 24 and an end effector driver.

At step 266, the robotic end effector positioning device 24 is positioned in a scanning region of the CT scanning device 44 so that a fiducial component 68 disposed on the end effector driver 70 can be scanned by the CT scanning device 44.

At step 268, the CT scanning device 44 performs a scan of the fiducial component 68 to generate scanning data. The CT scanning device control computer 42 generates a second plurality of digital images of the fiducial component 68 based on the scanning data.

At step 270, the CT scanning device control computer 42 transmits the second plurality of digital images to the robot control computer 46.

At step 272, the robot control computer 46 determines a position of the fiducial component 68 in the digital image coordinate system.

At step 274, the robot control computer 46 determines a first coordinate transformation matrix for transforming coordinates in the digital image coordinate system to coordinates in an end effector coordinate system based on: (i) the position of the fiducial component 68 in the end effector coordinate system, and (ii) the position of the fiducial component 68 in the digital image coordinate system. The first-quarter transformation matrix allows the robot control computer 46 to determine the location of the end effector 26 in the digital image coordinate system.

At step 276, the robot control computer 46 determines a second coordinate transformation matrix for transforming coordinates in the end effector coordinate system to coordinates in a robot coordinate system based on the robot kinematics properties.

At step 278, the robot control computer 46 determines a third coordinate transformation matrix for transforming coordinates in the digital image coordinate system to coordinates in the robot coordinate system based on the first and second coordinate transformation matrices. It should be understood, the when the robot control computer 46 can determine the location of the end effector 26 in the digital image coordinate system and the robot coordinate system, that the computer 46 can transform coordinates between the digital image coordinate system and the robot coordinate system.

At step 280, the robot control computer 46 determines a trajectory path in the robotic coordinate system by transforming the trajectory path specified in the digital image coordinate system via the third coordinate transformation matrix.

At step 282, the robotic end effector positioning device 24 holding the end effector 26 is moved such that the tip of end effector 26 is placed at the skin entry position and orientated coincident with the predetermined trajectory path.

At step 284, the respiratory monitoring computer 40 makes a determination as to whether the monitored respiratory state of the person is equal to a predetermined respiratory state. In particular, the respiratory monitoring computer 40 determines when the signal 135 is within a predetermined respiratory range ΔR. When the computer 40 determines the signal 135 is within the predetermined respiratory range, the computer 40 generates a gating signal 137 that is transmitted to the robot control computer 46. When the value of step 284 equals “yes”, the method advances to step 286. Otherwise, the method returns to step 284.

At step 286, the robot control computer 46 calculates a target position coordinate in the robot coordinate system.

At step 288, the robot control computer 46 induces the end effector driver 70 to move the tip of the end effector 26 toward the target position coordinate when an operator activates a joystick 47 and the monitored respiratory state equals the predetermined respiratory state.

At step 290, an operator makes a determination as to whether the tip of the end effector 26 has reached a target position by viewing a “real-time” digital image of the end effector 26 in the patient. Alternately, the robot control computer 46 could automatically make the determination as to whether the tip of the end effector 26 has reached the target position. When the value of step 290 equals “yes”, the method advances to the step 300. Otherwise, the method returns to step 284.

At step 300, the robot control computer 46 stops linear movement of the end effector 26.

The system and method for guiding an end effector to a target position within the person represents a substantial advantage over other systems. In particular, the system provides a technical effect of moving the end effector along a determined trajectory path within the person only when the person is within a predetermined respiratory state to obtain more accurate placement of the end effector toward the target location.

While embodiments of the invention are described with reference to the exemplary embodiments, it will be understood by those skilled in the art that various changes may be made and equivalence may be substituted for elements thereof without departing from the scope of the invention. In addition, many modifications may be made to the teachings of the invention to adapt to a particular situation without departing from the scope thereof. Therefore, it is intended that the invention not be limited to the embodiment disclosed for carrying out this invention, but that the invention includes all embodiments falling with the scope of the intended claims. Moreover, the use of the term's first, second, etc. does not denote any order of importance, but rather the term's first, second, etc. are used to distinguish one element from another. Furthermore, the use of the terms a, an, etc. do not denote a limitation of quantity, but rather denote the presence of at least one of the referenced items.

1. A method for guiding an end effector to a target position within a person, comprising:
generating a plurality of digital images of an interior anatomy of the person when the person has a predetermined respiratory state;
indicating a skin entry position on at least one of the digital images;
indicating the target position on at least one of the digital images;
determining a trajectory path based on the skin entry position and the target position; and
moving the end effector along the trajectory path toward the target position when the person has substantially the predetermined respiratory state.

2. The method of claim 1, wherein generating the plurality of digital images comprises:
moving the person within a scanning device along an axis; and,
generating the plurality of cross-sectional digital images during the movement wherein each cross-sectional image is generated at a distinct axial position.

3. The method of claim 1, wherein moving the end effector comprises:
monitoring a respiratory state of the person over time; and
moving the end effector along the trajectory path when a difference between the monitored respiratory state and the predetermined respiratory state is less than or equal to a threshold value.

4. The method of claim 1, wherein the end effector is moved at a predetermined speed.

5. The method of claim 1, wherein the plurality of digital images comprises a plurality of computerized tomography images.

6. A system for guiding an end effector to a target position within a person, comprising:
a respiratory monitoring device for monitoring a respiratory state of the person;
a scanning device configured to scan an interior anatomy of the person when the person has a predetermined respiratory state to generate scanning data;
a first computer generating a plurality of digital images based on the scanning data;
a second computer configured to display the plurality of digital images, the second computer further configured to allow an operator to indicate a skin entry position on at least one of the digital images, the second computer further configured to allow the operator to indicate the target position on at least one of the digital images, the second computer further configured to determine a trajectory path based on the skin entry position and the target position; and
an end effector insertion device having the end effector adapted to be inserted into the person, the second computer inducing the end effector insertion device to move the end effector along the trajectory path toward the target position when the person has substantially the predetermined respiratory state.

7. The system of claim 6, wherein the respiratory monitoring device comprises an infrared respiratory measurement device that detects a position of a chest of the person.

8. The system of claim 6, wherein the scanning device comprises a computerized tomography scanner and the plurality of digital images comprise a plurality of computerized tomography images.

9. The system of claim 6, wherein the end effector insertion device comprises an end effector driver configured to linearly move the end effector.

10. The system of claim 6, further comprising a positioning device operably coupled to the end effector insertion device for disposing the end effector insertion device at a predetermined position.

11. The system of claim 6, wherein the end effector insertion device can orient the end effector along the trajectory path.

12. The system of claim 6, wherein the second computer is further configured to move the person within the scanning device for generating the plurality of digital images during the movement wherein each digital image is generated at a distinct axial position of the person.

13. The system of claim 6, wherein the person has substantially the predetermined respiratory state when a difference between the monitored respiratory state and the predetermined respiratory state is less than or equal to a threshold value.

14. The system of claim 6, wherein the second computer induces the end effector insertion device to move the end effector along the trajectory path toward the target position at a predetermined speed.

15. A system for guiding an end effector to a target position within a person, comprising:
a respiratory monitoring device for monitoring a respiratory state of the person;
a scanning device configured to scan an interior anatomy of the person when the person has a predetermined respiratory state to generate scanning data;
a first computer generating a plurality of digital images based on the scanning data, the first computer further configured to display the plurality of digital images, the first computer further configured to allow an operator to indicate a skin entry position on at least one of the digital images, the first computer further configured to allow the operator to indicate the target position on at least one of the digital images, the first computer further configured to determine a trajectory path based on the skin entry position and the target position; and
an end effector insertion device having the end effector adapted to be inserted into the person, the first computer inducing the end effector insertion device to move the end effector along the trajectory path toward the target position when the person has substantially the predetermined respiratory state.

16. An article of manufacture, comprising:
a computer storage medium having a computer program encoded therein for guiding an end effector to a target position within a person, the computer storage medium including:
code for displaying and generating a plurality of digital images of an interior anatomy of the person when the person has a predetermined respiratory state;
code for indicating a skin entry position on at least one of the digital images;
code for indicating the target position on at least one of the digital images;
code for determining a trajectory path based on the skin entry position and the target position; and
code for moving the end effector along the trajectory path toward the target position when the person has substantially the predetermined respiratory state.

17. The article of manufacture of claim 16, wherein the code for displaying the plurality of digital images comprises:
code for scanning a predetermined region of the person along an axis; and,
code for generating the plurality of digital images during the movement wherein each digital image is generated at a distinct axial position.

18. The article of manufacture of claim 16, wherein the code for moving the end effector comprises:
code for monitoring a respiratory state of the person over time; and
code for moving the end effector along the trajectory path when a difference between the monitored respiratory state and the predetermined respiratory state is less than or equal to a threshold value.

19. The article of manufacture of claim 16, wherein the computer storage medium further includes code for moving the end effector at a predetermined speed into the person.

20. The article of manufacture of claim 16, wherein the plurality of digital images comprises a plurality of computerized tomography images.

21. A method for guiding an end effector to a target position within a person, comprising:
monitoring a respiratory state of a person during at least one respiratory cycle; and
moving an end effector along a trajectory path toward the target position in the person when the person has substantially a predetermined respiratory state.
